(19) United States
(12) Patent Application Publication
(10) Pub. No.: US 2026/0161708 A1
(43) Pub. Date: Jun. 11, 2026

(54) METHOD AND APPARATUS WITH GRAPH STRUCTURE DATA PROCESSING

(71) Applicant: SAMSUNG ELECTRONICS CO., LTD., Suwon-si (KR)

(72) Inventors: Sungwon KIM, Suwon-si (KR); Hyohyeong KANG, Suwon-si (KR); Hogyeong KIM, Suwon-si (KR); Seongeon PARK, Suwon-si (KR); Jinwoo Park, Suwon-si (KR)

(73) Assignee: SAMSUNG ELECTRONICS CO., LTD., Suwon-si (KR)

(21) Appl. No.: 19/254,261

(22) Filed: Jun. 30, 2025

(30) Foreign Application Priority Data
Dec. 9, 2024 (KR) 10-2024-0181799

(51) Int. Cl. G06F 16/901; G06N 3/0455

(52) U.S. Cl. CPC G06F 16/9024; G06N 3/0455

(57) ABSTRACT

A processor-implemented method including extracting a feature from action data, the action data being in chronological order and included in a synthesis process, obtaining a vector set corresponding to the action data, based on embedding data of the feature extracted from the action data, generating graph structure data corresponding to the vector set, based on time information of the action data, and obtaining encoding data corresponding to the graph structure data.

### CROSS-REFERENCE TO RELATED APPLICATIONS

This application claims the benefit under 35 USC § 119 (a) of Korean Patent Application No. 10-2024-0181799, filed on Dec. 9, 2024, in the Korean Intellectual Property Office, the entire disclosure of which is incorporated herein by reference for all purposes.

### BACKGROUND

### 1. Field

The following description relates to a method and apparatus with graph structure data processing, and more particularly, to a method and apparatus with graph structure data processing corresponding to a synthesis process.

### 2. Description of Related Art

Technology for simulating substance processing or synthesis recipes is used to mimic various physical or chemical parameters in a digital environment. This may enable predictions of optimal processing conditions and synthesis results without requiring experiments and acquiring the data which may otherwise be required to efficiently conduct experiments. Recently, machine learning and big data analysis have combined to improve the prediction accuracy of simulations in various industrial fields, such as substance development, semiconductors, or pharmaceuticals. There is a desire to employ artificial intelligence-based simulation and data processing technology, such technologies for accurately predicting the properties of substances, to be employed in and to improve the efficiency of substance synthesis or processing.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a general aspect, here is provided a processor-implemented method including extracting a feature from action data, the action data being in chronological order and included in a synthesis process, obtaining a vector set corresponding to the action data, based on embedding data of the feature extracted from the action data, generating graph structure data corresponding to the vector set, based on time information of the action data, and obtaining encoding data corresponding to the graph structure data.

The generating the graph structure data may include determining a node corresponding to respective segments of action data and determining an edge between determined nodes, based on the time information of the action data.

The determined node may include a first node corresponding to first action data and a first node feature of the first node may include a vector corresponding to the first action data.

The determined edge may include a first edge, the first edge connecting a first node corresponding to first action data to a second node corresponding to second action data and the method may further include determining a first edge feature of the first edge by encoding a difference between first action time information of the first action data and second action time information of the second action data.

The determined edge may include a first edge, the first edge connecting a first node corresponding to first action data to a second node corresponding to second action data and the method may further include determining a first edge feature of the first edge by encoding a difference between a first action data feature of the first action data and a second action data feature of the second action data.

The obtaining the encoding data corresponding to the graph structure data may include obtaining a nearby node feature of a nearby node included in a nearby node radius and an updated feature of an updated node, the updated node being updated by reflecting a connected node feature of an edge connected to the nearby node, based on a graph convolution operation and obtaining the encoding data corresponding to the graph structure data, based on the updated node feature.

The obtaining the encoding data corresponding to the graph structure data may include obtaining a nearby node feature of a nearby node included in a nearby node radius and an updated feature of an updated node, the updated node being updated by reflecting a connected node feature of an edge connected to the nearby node according to weights, based on an attention network and obtaining the encoding data corresponding to the graph structure data, based on the updated node feature.

The method may include obtaining property data, the property data being a result from a prediction from the synthesis process, based on the encoding data corresponding to the graph structure data.

The method may include generating an extracted feature extracted from the action data, based on the encoding data corresponding to the graph structure data for nodes corresponding to respective segments of the action data.

The method may include converting the encoding data corresponding to the graph structure data for the nodes into Gaussian noise and restoring the Gaussian noise to the encoding data for the nodes.

The generating may be performed by a decoder trained to generate the extracted feature from the action data, based on the encoding data corresponding to the graph structure data for the nodes and the restoring may be performed by a diffusion model trained to restore the Gaussian noise to the encoding data for the nodes.

The trained diffusion model may be configured to generate a latent vector from the Gaussian noise and the trained decoder may be configured to generate a feature of the action data corresponding to the synthesis process from the latent vector generated in the trained diffusion model.

In a general aspect, here is provided an electronic device including processors configured to execute instructions, a memory storing the instructions, and an execution of the instructions configures the processors to extract a feature from action data, the action data being in chronological order and included in a synthesis process, obtain a vector set corresponding to the action data, based on embedding data of the feature extracted from the action data, generate graph structure data corresponding to the vector set, based on time information of the action data, and obtain encoding data corresponding to the graph structure data.

The generating the graph structure data may include determining a node corresponding to respective segments of action data and determining an edge between determined nodes, based on the time information of the action data.

The determined node may include a first node corresponding to first action data and a first node feature of the first node may be a vector corresponding to the first action data.

The determined edge may be a first edge, the first edge connecting a first node corresponding to first action data to a second node corresponding to second action data and the processors may be further configured to determine a first edge feature of the first edge by encoding a difference between first action time information of the first action data and second action time information of the second action data.

The determined edge may include a first edge, the first edge connecting a first node corresponding to first action data to a second node corresponding to second action data and the processors may be further configured to determine a first edge feature of the first edge by encoding a difference between a first action data feature of the first action data and a second action data feature of the second action data.

The obtaining the encoding data corresponding to the graph structure data may include obtaining a nearby node feature of a nearby node included in a nearby node radius and an updated feature of an updated, the updated node being updated by reflecting a connected feature of an edge connected to the nearby node, based on a graph convolution operation and obtaining the encoding data corresponding to the graph structure data, based on the updated node feature.

The processors may be further configured to obtain property data, the property data being a result from a prediction from the synthesis process, based on the encoding data corresponding to the graph structure data.

The processors may be further configured to generate an extracted feature extracted from the action data, based on the encoding data corresponding to the graph structure data for nodes corresponding to respective segments of the action data.

Other features and aspects will be apparent from the following detailed description, the drawings, and the claims.

### BRIEF DESCRIPTION OF THE DRAWINGS

FIG. 1 illustrates an example method with graph structure data processing corresponding to a synthesis process according to one or more embodiments.

FIG. 2 illustrates an processing method according to one or more embodiments.

FIGS. 3A and 3B illustrate example feature data output from action data according to one or more embodiments.

FIGS. 4A and 4B illustrate example methods of generating an embedding vector of a feature extracted from action data according to one or more embodiments.

FIG. 5 illustrates an example generation of graph structure data according to one or more embodiments.

FIG. 6 illustrates an example convolution operation of graph structure data according to one or more embodiments.

FIG. 7 illustrates an example graph encoder according to one or more embodiments.

FIG. 8 illustrates an example method with a property data prediction model according to one or more embodiments.

FIG. 9 illustrates an example autoencoder with graph structure data processing according to one or more embodiments.

FIG. 10 illustrates an example autoencoder with a diffusion model for generating features of new action data according to one or more embodiments.

FIGS. 11A and 11B illustrate an examples of an autoencoder with a diffusion model for generating features of new action data according to one or more embodiments.

FIG. 12 illustrates an example electronic device according to one or more embodiments.

Throughout the drawings and the detailed description, unless otherwise described or provided, the same or like drawing reference numerals may be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when a component or element is described as being “on”, “connected to,” “coupled to,” or “joined to” another component, element, or layer it may be directly (e.g., in contact with the other component or element) “on”, “connected to,” “coupled to,” or “joined to” the other component, element, or layer or there may reasonably be one or more other components, elements, layers intervening therebetween. When a component or element is described as being “directly on”, “directly connected to,” “directly coupled to,” or “directly joined” to another component or element, there can be no other elements intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

As used herein, the term “and/or” includes any one and any combination of any two or more of the associated listed items. The phrases “at least one of A, B, and C”, “at least one of A, B, or C”, and the like are intended to have disjunctive meanings, and these phrases “at least one of A, B, and C”, “at least one of A, B, or C”, and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning.

Due to manufacturing techniques and/or tolerances, variations of the shapes shown in the drawings may occur. Thus, the examples described herein are not limited to the specific shapes shown in the drawings, but include changes in shape that occur during manufacturing.

Unless otherwise defined, all terms, including technical and scientific terms, used herein have the same meaning as commonly understood by one of ordinary skill in the art to which this disclosure pertains and based on an understanding of the disclosure of the present application. Terms, such as those defined in commonly used dictionaries, are to be interpreted as having a meaning that is consistent with their meaning in the context of the relevant art and the disclosure of the present application and are not to be interpreted in an idealized or overly formal sense unless expressly so defined herein. The use of the term “may” herein with respect to an example or embodiment, e.g., as to what an example or embodiment may include or implement, means that at least one example or embodiment exists where such a feature is included or implemented, while all examples are not limited thereto.

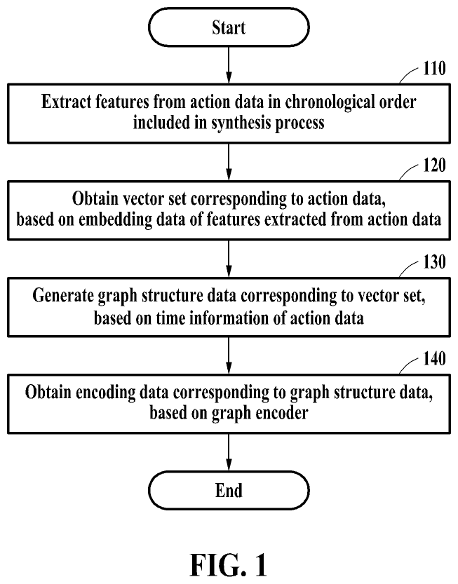

FIG. 1 illustrates an example method with graph structure data processing corresponding to a synthesis process according to one or more embodiments.

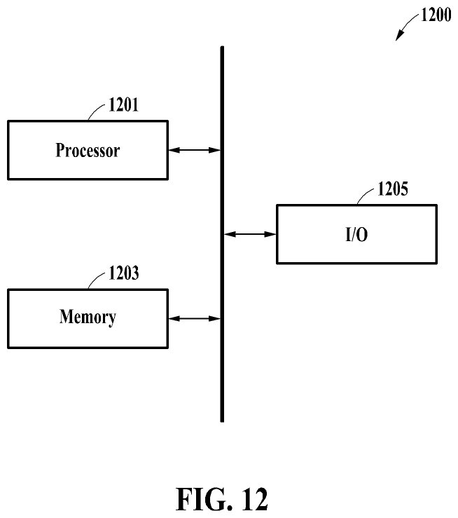

The graph structure data processing method corresponding to a synthesis process may be performed in an electronic device including at least one processor (e.g., processor 1201 of electronic apparatus 1200 of FIG. 12). The specific hardware configuration of the electronic device that performs the graph structure data processing method corresponding to a synthesis process is described below. Hereinafter, the graph structure data processing method corresponding to a synthesis process may be briefly referred to as the graph structure data processing method.

Referring to FIG. 1, in a non-limiting example, the graph structure data processing method may include operation 110 of extracting features from action data in chronological order included in the synthesis process.

The synthesis process is data that instructs a process, a procedure, or a recipe for synthesizing a substance. For example, the synthesis process may include one or more of text data that instructs the process of synthesizing a substance and graph data that instructs changes in elements or conditions for synthesizing a substance over time.

In an example, the synthesis process may include segments of action data. Action data is data that instructs actions to adjust at least one condition. For example, the action data may include data that instructs actions, such as changing the temperature of an experimental environment, changing the gas composition of the experimental environment, adding a substance, or purifying.

A segment of action data may correspond to each step of the synthesis process. The chronological order may be determined between the segments of action data. For example, the action data may include information indicating action data corresponding to a previous sequence and/or information indicating action data corresponding to a subsequent sequence.

In an example, the action data may include time information. For example, the action data may include information indicating a start time. For example, the action data may include information indicating when the action data begins relative to the initiation of the synthesis process. For example, the action data may include the time information indicating start and end times. For example, the action data may include information indicating a duration for which the process or step takes place.

One or more features may be extracted from each action data segment. Features extracted from action data may include the attributes of information extracted from the action data and/or data that quantifies information included in the action data. For example, the features may include at least one of a feature regarding the type of action included in the action data, a feature regarding temperature at which the action included in the action data is performed, a feature regarding a condition indicated by the action data, a feature regarding a change direction of the condition indicated by the action data, a feature regarding a change amount of the condition indicated by the action data, and a feature regarding time information indicated by the action data.

In an example, operation 110 of extracting features from action data may include a step of extracting a predetermined type of feature from each action data segment. For example, the predetermined type of feature may include a categorical feature including at least one of an action type and a substance type. The categorical feature may be a feature that represents data as a finite number of categories or groups. The value of the categorical feature may be determined to be at least one of a plurality of labels included in a category. For example, the predetermined type of feature may include a numerical feature including at least one of quantity of substance, temperature, and duration. The numerical feature may correspond to a feature that represents data as numerical values. The value of the numerical feature may be determined to be numerical data.

As an example, a substance type feature may be divided into one or more sub-features. The substance type feature may be divided into sub-features including at least one of a cation-type feature, an anion-type feature, a solvent-type feature, and a concentration feature according to the chemical properties of a substance.

In an example, some types of features may not be extracted from any segment of action data. A value of a feature that is not extracted from the segment of action data may be determined to be a value (e.g., null) indicating that the feature is not extracted. Specific examples of the features extracted from each action data segment are described below.

In an example, operation 110 of extracting features from action data may include a step of extracting features from action data for each time step included in the synthesis process, which is time-series data. The synthetic process, which is time-series data, may include data indicating changes in conditions over time or data indicating conditions recorded at time steps of a certain time interval (e.g., 1 second, 1 minute, 10 minutes, etc.). The action data may include data indicating conditions recorded at a time step included in the synthesis process.

In an example, the graph structure data processing method may include operation 120 of obtaining a vector set corresponding to action data based on embedding data of features extracted from action data.

Feature(s) extracted from a segment of action data may be embedded in order to be generated as a vector of a certain size. In other words, a vector may be generated, the vector including the embedding data of feature(s) corresponding to each segment of action data. If n segments of action data correspond to the synthesis process, n vectors may be generated. A vector obtained by embedding features extracted from action data may be referred to as a vector corresponding to the action data, a vector of the action data, or an action data vector.

The vector set may include vectors respectively corresponding to the segments of action data. For example, if the synthesis process includes first action data, second action data, and third action data, the vector set for the synthesis process may be obtained that includes a first vector corresponding to the first action data, a second vector corresponding to the second action data, and a third vector corresponding to the third action data.

In an example, operation 120 of obtaining a vector sequence may include a step of obtaining the embedding data of features extracted from action data, based on preprocessing of the features extracted from the action data.

As an example, the preprocessing of features may include determining the value of an unextracted feature. As described above, the types of features predetermined in the action data may include an unextracted type of feature. In feature preprocessing, the value of an unextracted feature may be determined to be a preset value (e.g., −1).

As an example, the feature preprocessing may include normalizing numerical features. The scales of different types of numerical features may be transformed into a certain range of scales through normalization. For example, the numerical features may be normalized to be real numbers greater than or equal to 0 and less than or equal to 1.

As an example, the feature preprocessing may include encoding categorical features. Each category of the categorical features may be mapped to a numerical value. The value of a categorical feature may be changed to the mapped numerical value. For example, if the value of a categorical feature is determined as any one of A, B, and C, A may be mapped to 0, B to 1, and C to 2. If the value of the categorical feature is A, the value of the categorical feature may be changed to 0 mapped to A through preprocessing.

In an example, operation 120 of obtaining a vector sequence may include obtaining feature-specific embedding data extracted from action data, aggregating the feature-specific embedding data to generate a vector corresponding to each segment of the action data, and obtaining the vector sequence including vectors respectively corresponding to segments of the action data.

In an example, a step of generating a vector corresponding to each action data segment may include generating a first aggregate feature by aggregating embedding data corresponding to at least some of features extracted from each action data segment, generating a second aggregate feature by aggregating the embedding data corresponding to at least some of the features extracted from each action data segment, and aggregating the first aggregate feature and the second aggregate feature.

The process of aggregating feature-specific embedding data to generate a vector is described in greater detail below.

In an example, the graph structure data processing method may include operation 130 of generating graph structure data corresponding to vectors based on the time information of action data.

The graph structure data may correspond to a data structure including nodes (or vertices) and edges (or lines) connecting the nodes. As an example, the graph structure data may include the nodes corresponding to the vectors, respectively. A node included in the graph structure data may correspond to a action data segment. Feature(s) of the node included in the graph structure data may be determined to be a vector corresponding to the action data segment. As an example, if the node includes a first node corresponding to the first action data, a feature of the first node may include a vector corresponding to the first action data.

In an example, operation 130 may include determining a node that corresponds to each action data segment and determining an edge between the determined nodes based on the time information of the action data. Based on the time information, a neighboring node that is to be connected to each node by an edge may be determined. A neighboring node of a specific node may refer to a node connected to the node by an edge. As an example, if a difference between the start times of the first action data and second action data is within a predetermined time range (e.g., 10 minutes), a node corresponding to the first action data and a node corresponding to the second action data may be determined to be connection nodes to each other.

In an example, operation 130 may include determining a node that corresponds to each vector and determining an edge between the determined nodes based on order information from among the segments of action data. As an example, the synthesis process may be a series of sequences of segments of action data. A first particular node of action data may be connected by an edge to another particular node of action data, where, in an example, the other particular may immediately follow the first particular node in chronological order and/or other respective nodes of action data immediately preceding it in chronological order in the synthesis process.

Features of an edge may be determined based on features of action data corresponding to a node connected by the edge. As an example, the features of the edge may be determined based on differences between at least some of the features of the action data.

As an example, if the determined edge includes a first edge connecting the first node corresponding to the first action data to a second node corresponding to the second action data, the generating of the graph structure data may include determining features of the first edge by encoding differences between features of the first action data and features of the second action data. For example, the generating of the graph structure data may include determining the features of the first edge by encoding a difference between the time information of the first action data and the time information of the second action data. For example, the generating of the graph structure data may include determining the features of the first edge by encoding a difference between an action type of the first action data and an action type of the second action data. For example, the generating of the graph structure data may include determining the features of the first edge by encoding a difference between a temperature at which an action of the first action data is performed and a temperature at which an action of the second action data is performed.

Specific examples of the graph structure data corresponding to the vector set are described in greater detail below.

In an example, the graph structure data processing method may include operation 140 of obtaining encoding data corresponding to the graph structure data based on a graph encoder. The encoding data corresponding to the graph structure data is data obtained by compressing or converting the graph structure data. For example, the encoding data corresponding to the graph structure data may include data obtained by encoding features of nodes and features of edges included in the graph structure data. As an example, the encoding data corresponding to the graph structure data may include an embedding vector or a latent vector of the graph structure data obtained based on the graph encoder. The graph encoder is described in greater detail below.

In an example, operation 140 may include obtaining features of an updated node where is updated node is updated by reflecting features of a nearby node included in a nearby node radius and features of an edge connected to the nearby node based on a graph convolution operation. Thus, the encoding data corresponding to the graph structure data may be obtained based on the features of the updated node.

Based on a graph convolution operation, features of a node may be updated by combining the features of the node with features of a nearby node. Features of each node included in the graph structure data may be updated by reflecting features of a nearby node.

A value of the nearby node radius may be predetermined to be a value relative to an edge. For example, the nearby node radius may be determined based on the number of edges and the length of the edge(s).

For example, if the nearby node radius is set to two edges, node(s) connected to a node through two edges may be determined to be the nearby node of that node. In other words, a first connection node connected to a node by an edge and a second connection node connected to the first connection node by the edge may be determined to be the nearby node of that node.

As an example, the nearby node radius may be determined to be a value of edge length. The length of an edge in the graph structure data may be determined by a similarity between nodes connected by the edge. In an example, the length of an edge in the graph structure data may be determined by the similarity or distance between two vectors corresponding to two nodes connected by the edge. For example, the length of an edge in the graph structure data may be determined by Euclidean distance between two vectors corresponding to two nodes connected by the edge. Nodes included within the edge length that are determined by the nearby node radius of a node may be determined to be nearby nodes of that node. The determining of the nearby nodes is described in greater detail below.

In an example, in operation 140 features of a node updated may be obtained by reflecting features of a nearby node included in the nearby node radius and features of an edge connected to the nearby node according to weights based on a graph attention network (GAT) and may also include obtaining the encoding data corresponding to the graph structure data based on the updated features of the node.

In an example, the graph structure data processing method may include obtaining property data that may be predicted as a finding from the synthesis process based on the encoding data corresponding to the graph structure data. As an example, the graph encoder may be included in a property data prediction model. The property data prediction model may output the property data predicted as a finding from the synthesis process based on the encoding data corresponding to the graph structure data. The property data prediction model is described in greater detail below.

In an example, the graph structure data processing method may include training a decoder to generate features extracted from action data based on node-specific encoding data corresponding to the graph structure data. The graph encoder that generates the node-specific encoding data corresponding to the graph structure data may correspond to an encoder of an autoencoder for processing the graph structure data, and the decoder trained to generate the features extracted from action data may correspond to a decoder of the autoencoder for processing the graph structure data.

In an example, the graph structure data processing method may include converting the node-specific encoding data corresponding to the graph structure data into Gaussian noise and training a diffusion model to restore the converted Gaussian noise into the node-specific encoding data. The trained diffusion model may generate latent vectors from the Gaussian noise. The trained decoder may generate the features of action data corresponding to a synthesis process from the latent vectors generated by the trained diffusion model.

The autoencoder for processing the graph structure data is described in greater detail below.

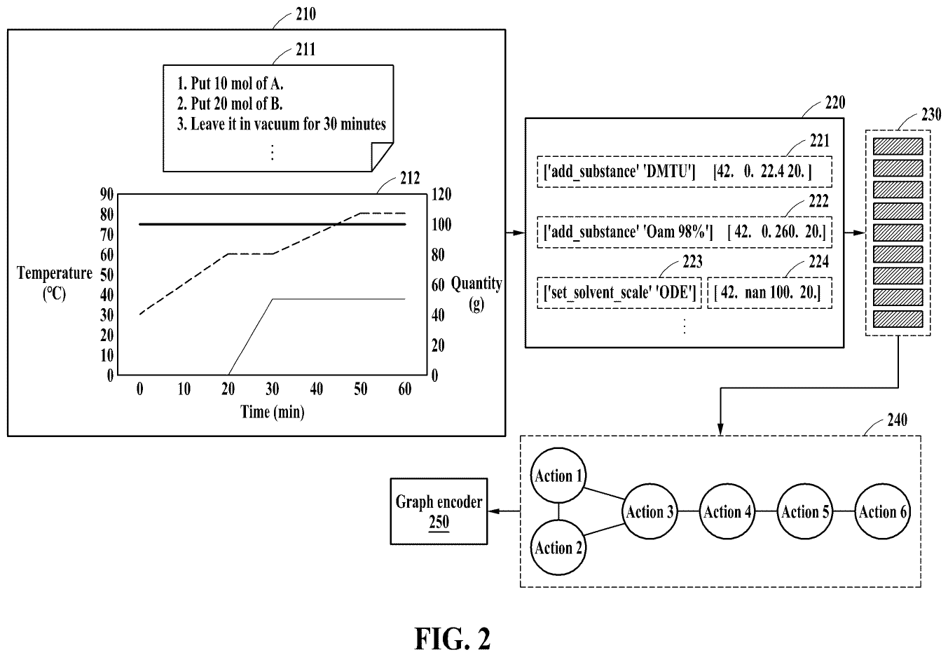

FIG. 2 illustrates an processing method according to one or more embodiments.

Referring to FIG. 2, in a non-limiting example, synthesis process data 210 may be input data of an electronic device (e.g., electronic device 1200 of FIG. 12) configured to perform the graph structure data processing method. As an example, the synthesis process data 210 may include text data 211 and/or graph data 212. The text data 211 and the graph data 212 are illustrated examples of the synthesis process data 210, however, the synthesis process data 210 is not limited thereto.

The text data 211 may include data that expresses each step of a synthesis process as text. The text data 211 may include data expressed in natural language or data with text being input to a predetermined format (e.g., a table). Some texts separated by steps in the text data 211 may correspond to action data.

The graph data 212 may include data indicating changes in conditions for substance synthesis over time. As an example, the graph data 212 may include graph data indicating temperature changes over time. As an example, the graph data 212 may include data indicating changes in the quantity of substance over time. Action data may correspond to data indicating a state of a time step in the graph data 212.

In an example, feature set data 220 may be extracted from action data in a chronological order of the synthesis process. A feature set may correspond to one action data segment. As an example, a first feature set 221 may include features extracted from first action data, and a second feature set 222 may include features extracted from second action data.

As an example, the feature set data 220 may include a vector 223 listing categorical features and a vector 224 listing numerical features, in which the categorical and numerical features are extracted from each action data segment. A feature set may include categorical features including action types and substance types. A feature set may include numeric features including time, duration, quantity of substance, and temperature.

In an example a vector set 230 corresponding to the action data may be obtained based on embedding data of features extracted from the action data. The vector set 230 may include a vector that embeds a feature set extracted from each action data segment. As an example, the vector set 230 may include vectors in which the feature set extracted from each action data segment is embedded with a predetermined size.

Based on the time information of action data, graph structure data 240 corresponding to the vector set 230 may be generated. The generating of the graph structure data 240 is described in greater detail below.

The graph structure data 240 may be applied to a graph encoder 250. Encoding data corresponding to the graph structure data 240 may be generated by the graph encoder 250.

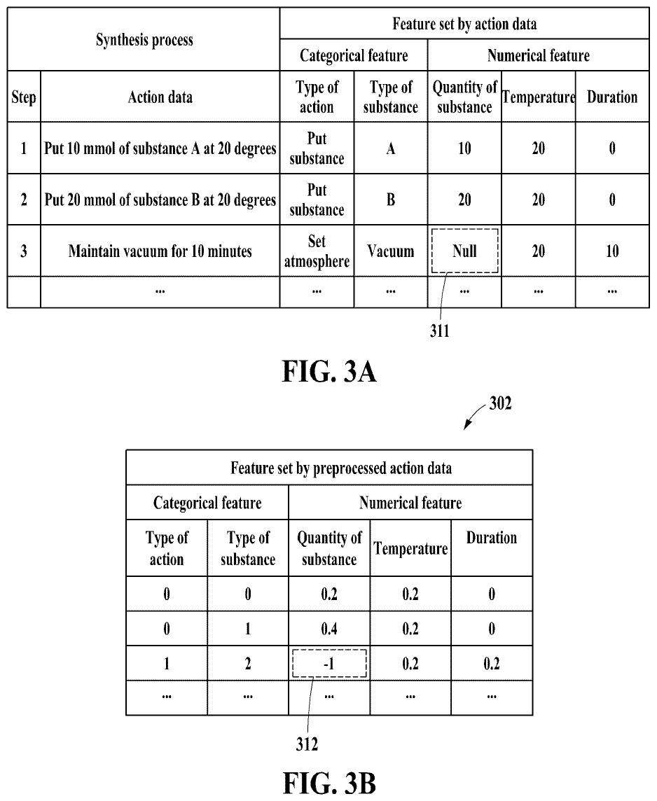

FIGS. 3A and 3B illustrate example feature data output from action data according to one or more embodiments.

Referring to FIG. 3A, in a non-limiting example, a feature set including a plurality of features may be extracted from each segment of action data included in a synthesis process. As an example, the features may be extracted based on keywords extracted from the action data. As an example, the features may be extracted from the action data by using a generative model. The method for extracting features from action data is not limited to the foregoing examples, and various methods may be used.

For example, a feature set including an action type, a substance type, the quantity of substance, a temperature, and duration may be extracted from each segment of action data.

The action type and the substance type may belong to categorical features. The value of an action type feature may be determined by a predetermined label, like ‘Put substance’, ‘Set atmosphere’, or ‘Set temperature’. The value of a substance type feature may be determined by an identification value (e.g. a name or a chemical formula) of substance, like ‘A’, ‘B’, ‘vacuum’, or ‘nitrogen’.

The quantity of substance, temperature, and duration may be numerical features. The values of numerical features may be determined to be numerical data, like natural numbers, integers, or real numbers.

In an example, some types of features may not be extracted from any segment of action data. For example, a feature 311 of substance quantity type may not be extracted from third action data. The value of an unextracted feature may be determined to be a value (e.g., null) indicating that the feature is not extracted.

The feature set of FIG. 3A may be preprocessed to be converted into feature set data 302 as illustrated in FIG. 3B.

Referring to FIG. 3B, in a non-limiting example, a value of a feature 311 having a null value may be converted into a predetermined value (e.g., −1) 312 through preprocessing. As an example, the numerical features may be normalized to have real numbers greater than or equal to 0 and less than or equal to 1. As an example, values of categorical features may be changed to mapped numerical values. For example, among the feature values of the action type, ‘Put substance’ may be changed to 0, and, among the feature values of the action type, ‘Set to standby’ may be changed to 1.

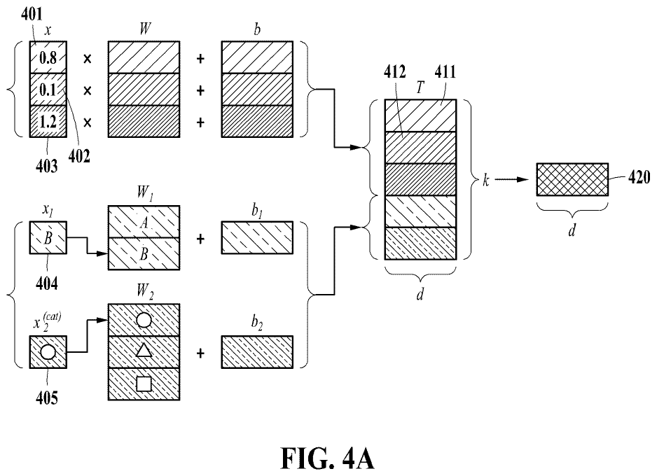

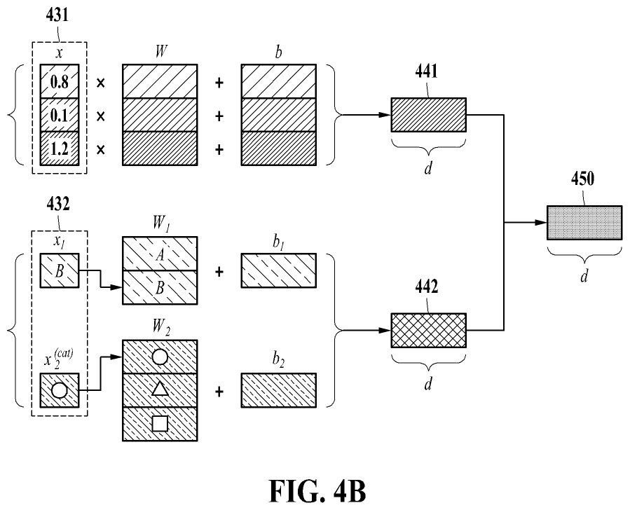

FIG. 4A illustrates an example method of generating an embedding vector of a feature extracted from action data according to one or more embodiments.

Referring to FIG. 4A, in a non-limiting example, embedding data may be generated from each feature. For example, first embedding data 411 may be embedding data that is generated from a first feature 401, and second embedding data 412 may be embedding data that is generated from a second feature 402.

In an example, in a where the features are numerical features, embedding data may be generated by multiplying a value x of each feature by a weight W and adding a bias b thereto. Weights and biases may be determined for each feature. As an example, a weight multiplied by the first feature 401 may be different from a weight multiplied by the second feature 402. As an example, a bias added to the first feature 401 may be different from a bias added to the second feature 402.

In an example, in the case of categorical features, the value of each feature may be changed to a mapped weight, and embedding data may be generated by adding a bias to the weight. Weights and biases may be determined for each feature. As an example, the weight mapped to a fourth feature 404 may be different from the weight mapped to a fifth feature 405. As an example, a bias added to the fourth feature 404 may be different from a bias added to the fifth feature 405.

The respective embedding data of different features may be generated as embedding data of the same size. For example, the embedding data of the features 401, 402, 403, 404, and 405 extracted from the action data may be generated with a same size of 1 Xd.

The embedding data obtained from each feature may be aggregated to be generated as a vector 420 having a size corresponding to a size the action data. For example, the aggregating of the embedding data obtained from each feature may be performed by using various aggregation methods, including a sum, weighted sum, mean, weighted average, self-attention, or max pooling, of the embedding data obtained from each feature.

FIG. 4B illustrates an example method of generating an embedding vector of a feature extracted from action data according to one or more embodiments. Referring to FIG. 4B, in a non-limiting example, a first aggregate feature 441 may be generated by aggregating embedding data corresponding to a portion of the features extracted from action data. A second aggregate feature 442 may be generated by aggregating embedding data corresponding to a portion of other features from among the features extracted from the action data.

In an example, the first aggregate feature 441 may be generated by aggregating embedding data of features 431 belonging to numerical features. The second aggregate feature 442 may be generated by aggregating embedding data of features 432 belonging to the categorical feature.

A vector 450, which is the embedding data of the features extracted from the action data, may be generated by aggregating the first aggregate feature 441 and the second aggregate feature 442.

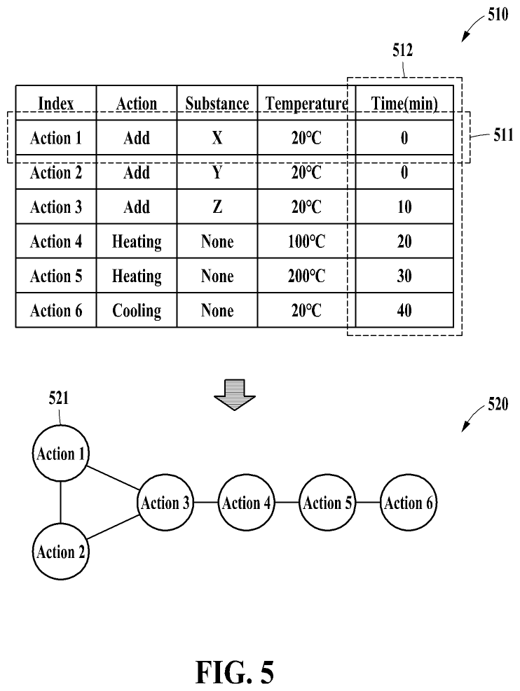

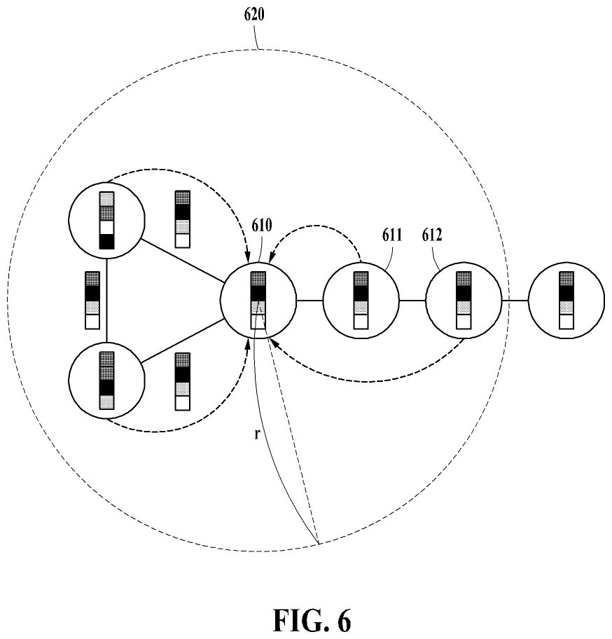

FIG. 5 illustrates an example generation of graph structure data according to one or more embodiments.

Referring to FIG. 5, in a non-limiting example, a feature set 510 including a plurality of features may be extracted from each segment of action data included in a synthesis process. As described above, based on embedding data of the feature set 510 extracted from segments of action data, a vector set including a vector corresponding to each action data segment may be obtained.

Based on time information included in the feature set 510 of the action data, graph structure data 520 corresponding to the vector set may be generated.

Each node of the graph structure data 520 may correspond to each action data segment. Features of each node of the graph structure data 520 may be determined to be vectors corresponding to the features extracted from each action data segment. For example, a first node 521 may correspond to first action data having an index of Action 1, and features of the first node 521 may correspond to vectors of the first action data obtained by embedding features 511 extracted from the first action data.

Time information included in the feature set 510 of the action data may include start time information 512 of the action data. The start time information 512 may include information indicating the time at which the action data begins based on the initiation of the synthesis process. For example, if the start time information of the first action data is 0 minutes, the first action data may begin simultaneously with the initiation of the synthesis process. For example, if the start time information of third action data whose index is Action 3 is 10 minutes, the third action data may start 10 minutes after the initiation of the synthesis process.

Edges connecting nodes of the graph structure data 520 may be determined based on a difference in start times between segments of action data. For example, if a difference between the start time of the first action data and the start time of second action data whose index is Action 2 is less than a predetermined time range (e.g., 10 minutes), a node corresponding to the first action data and a node corresponding to the second action data may be determined to be connection nodes of each other. In other words, the edge between the node corresponding to the first action data and the node corresponding to the second action data may be determined. For example, if a difference between the start time of the first action data and a start time of fourth action data whose index is Action 4 exceeds the predetermined time range (e.g., 10 minutes), the node corresponding to the first action data and a node corresponding to the fourth action data are not determined as connection nodes, and an edge between the node corresponding to the first action data and the node corresponding to the fourth action data may not be generated.

Features of an edge may be determined based on features of action data corresponding to a node connected by the edge. In an example, the features of the edge may be determined based on differences between at least some of the features of the action data. As described above, features of an edge may be determined to be embedding vectors obtained by encoding at least one of, for example, a difference in start times between segments of action data corresponding to two connection nodes, a difference in action types, and a difference in temperatures.

FIG. 6 illustrates an example convolution operation of graph structure data according to one or more embodiments.

Referring to FIG. 6, in a non-limiting example, a nearby node radius may be determined to be the length of an edge for the graph convolution operation. For example, the nearby node radius may be determined to be an edge length r, and nodes within the nearby node radius from a reference node may be determined to be the nearby nodes of the reference node.

For example, if a first node 610 included in the graph structure data is referred to as the reference node, node(s) included in a nearby node radius 620, which is the edge length r, centered on the first node 610 may be determined to be the nearby nodes of the first node 610. In other words, the nearby nodes of the first node 610 may include connection nodes connected to the first node 610 by an edge having an edge length of r or less. The nearby nodes of the first node 610 may include nodes connected to the first node 610 by two or more edges whose sum of edge lengths is less than or equal to the edge length r as well as connection node(s) directly connected to the first node 610 by one edge. For example, if the sum of the edge lengths of the first node 610 and a second node 611 and the edge lengths of the second node 611 and a third node 612 are less than or equal to the edge length r, the third node 612 may be determined to be a nearby node of the first node 610.

The graph convolution operation may include updating features of the reference node by applying features of nearby nodes to the features of the reference node. By means of the graph convolution operation, the features of the reference node may be updated by reflecting the features of the nearby nodes. Through the graph convolution operation, features of each node included in the graph structure data may be updated by reflecting features of the nearby nodes.

In an example, if the features of the nearby nodes are reflected in the reference node, the features of the reference node may be updated based on weights corresponding to the nearby nodes. As an example, weights between nodes may be determined based on an attention function defined to calculate importance between the nodes. Based on the weights determined for each nearby node of the reference node, the extent to which the features of the nearby nodes are reflected in the features of the reference node may be determined.

Features of nearby nodes with high weights may be reflected in the features of the reference node at a higher reflection rate than features of nearby nodes with low weights. The operation of updating the features of the reference node based on the weights of the nearby nodes may be performed based on a GAT.

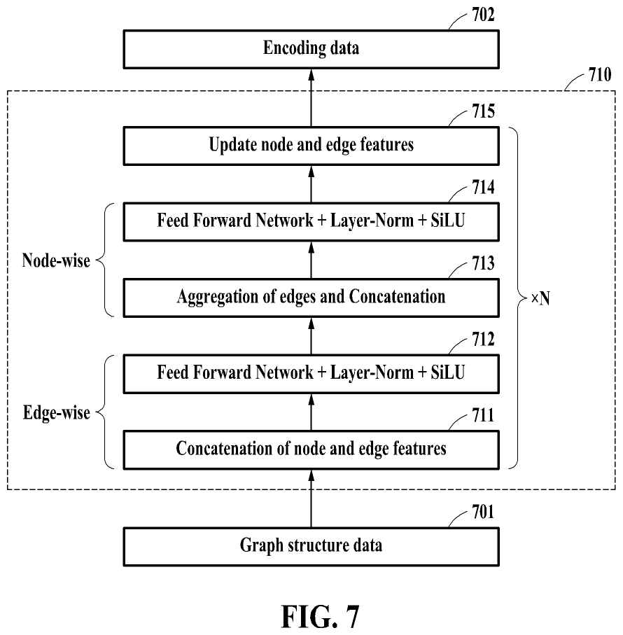

FIG. 7 illustrates an example graph encoder according to one or more embodiments.

Referring to FIG. 7, in a non-limiting example, a graph encoder 710 may be a model that receives graph structure data 701 as an input and outputs encoding data 702 of the graph structure data 701.

In an example, the graph encoder 710 may perform a graph convolution operation to update features of nodes and features of edges included in the graph structure data 701. The graph convolution operation may include operations for edgewise processing and operations for node-wise processing.

More specifically, the operations for edgewise processing may include concatenation 711 of node and edge features. Concatenation 711 of node and edge features may include connecting features of edges to features of nodes connected to the edges by each edge included in the graph structure data 701. For example, if a first node and a second node are connected to a first edge, features of the first edge, the first node, and the second node may be connected. The operations for edgewise processing may include obtaining the embedding data of connected features by applying the connected features to Feed Forward Network+Layer-Norm+SiLU layer 712. Feed Forward Network+Layer-Norm+SiLU layer 712 may include a feed forward network (FFN) for learning the connected features of nodes and edges obtained by concatenation 711 of node and edge features, a layer-norm for normalizing vectors output from the FFN, and a Sigmoid linear unit (SiLU), that is, an activation function. Features of each edge may be updated as an output of Feed Forward Network+Layer-Norm+SiLU layer 712.

In an example, the operations for node-wise processing may include aggregation 713 of edges and concatenation. Aggregation 713 of edges and concatenation may include aggregating features of edge(s) that connect nodes to the nearby nodes of the nodes by each node included in the graph structure data 701 and connecting the aggregated features to features of the nodes. As described above, the nearby nodes may be determined based on a nearby node radius. The operations for node-wise processing may include obtaining the embedding data of connected features by applying the connected features to Feed Forward Network+Layer-Norm+SiLU layer 714. Feed Forward Network+Layer-Norm+SiLU layer 714 may include a feed forward network FFN for learning the connected features of nodes and edges obtained by aggregation 713 of edges and concatenation, a layer-norm for normalizing vectors output from the FFN, and an SiLU, that is, an activation function. Features of each node may be updated as an output of Feed Forward Network+Layer-Norm+SiLU layer 714.

Features of nodes and edges included in the graph structure data may be updated in operation 715 through the graph convolution operation. The graph encoder 710 repeats the graph convolution operation N times such that the features of nodes and edges included in the graph structure data may be updated in operation 715.

The graph encoder 710 may output the encoding data 702 of the graph structure data 701 generated based on the updated features of nodes and edges. In an example, embedding vectors corresponding to the features of node(s) included in the graph structure data 701 may be output as the encoding data 702 of the graph structure data 701. For example, the embedding vectors of a certain size generated by aggregating the features of node(s) included in the graph structure data 701 may be output as the encoding data 702 of the graph structure data 701.

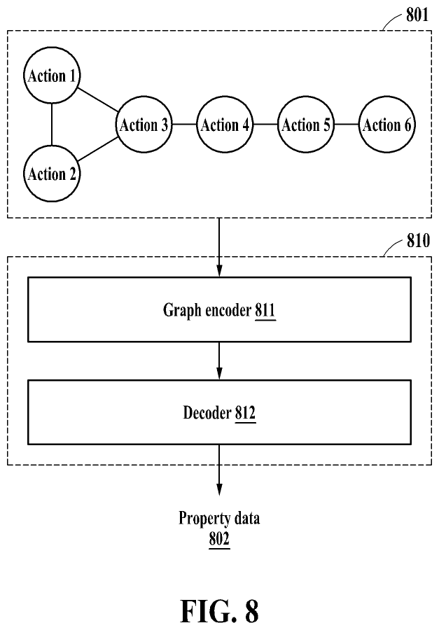

FIG. 8 illustrates an example method with a property data prediction model according to one or more embodiments.

Referring to FIG. 8, in a non-limiting example, a property data prediction model 810 may include a model trained to predict property data 802 as a finding from a synthesis process corresponding to graph structure data 801. That is, the property data 802 may be a result of a prediction from the synthesis process. Property is information indicating a quality or trait belonging to a substance. For example, the property may include at least one of quantum efficiency, synthetic yield, wavelength, quantum dot size, emission wavelength, and full width at half maximum (FWHM).

In an example, the property data prediction model 810 may include a graph encoder 811 and a decoder 812. As described above, the graph encoder 810 may be an encoder that generates encoding data of the graph structure data 801 from the graph structure data 801. For example, the graph encoder 811 may include the graph encoder 710 described above with reference to FIG. 7.

The decoder 812 may correspond to a regression head. The decoder 812 may be trained to predict the property data 802 from the encoding data of the graph structure data 801 output from the graph encoder 811. As an example, the encoding data of the graph structure data 801 may include embedding vectors of a certain size generated by aggregating features of node(s) of the graph structure data 801 updated by a graph convolution operation.

By using the property data prediction model 810 to obtain the property data 802 predicted as the finding from the synthesis process, the property data 802 of a substance predicted to be generated from the synthesis process may be obtained without an actual experiment on the synthesis process.

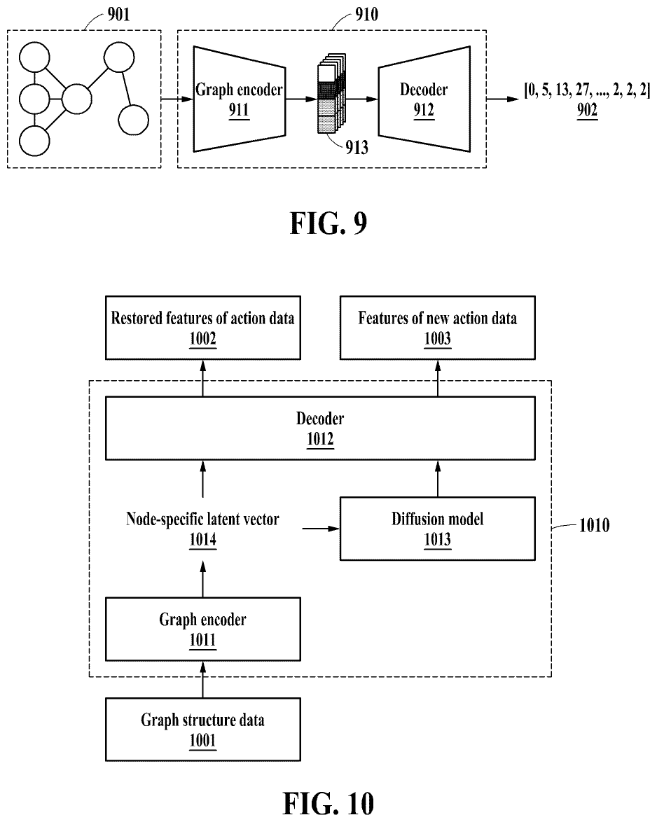

FIG. 9 illustrates an example autoencoder with graph structure data processing according to one or more embodiments.

Referring to FIG. 9, in a non-limiting example, an autoencoder 910 for processing graph structure data 901 may include a model trained to generate features 902 extracted from action data based on encoding data 913 obtained from the input graph structure data 901. For example, the features 902 extracted from the action data may correspond to the feature set data 220 extracted from the action data of FIG. 2.

The autoencoder 910 may include a graph encoder 911 and a decoder 912. As described above, the graph encoder 911 may be an encoder that generates the encoding data 913 of the graph structure data 901 from the graph structure data 901. For example, the graph encoder 911 may include the graph encoder 710 described above with reference to FIG. 7.

The decoder 912 may be trained to restore the features 902 extracted from the action data corresponding to the graph structure data 901 from the encoding data 913 of the graph structure data 901 output from the graph encoder 911. In an example, the encoding data 913 of the graph structure data 901 may include features of each node of the graph structure data 901 updated by a graph convolution operation. For example, the decoder 912 may be trained to restore the features 902 extracted from the action data including at least one of an action type, time information, the quantity of substance, and a temperature corresponding to each action data segment included in a synthesis process.

In an example, the decoder 912 may be trained to convert numerical features into categorical features and to output the converted features. In other words, the decoder 912 may be trained to output any one of predetermined discrete values rather than continuous real numbers as a value of a numerical feature. For example, when outputting a feature value of temperature, which is a numerical feature, the decoder 912 may be trained to output values (e.g., 0, 5, 10, 15, . . . , 100) in 5-degree increments from 0 to 100.

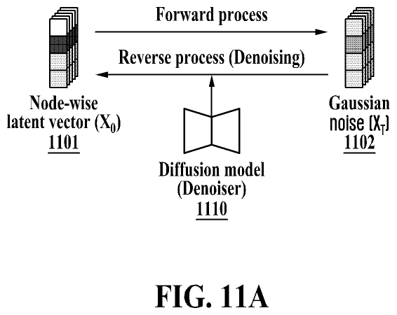

FIG. 10 illustrates an example autoencoder with a diffusion model for generating features of new action data according to one or more embodiments.

Referring to FIG. 10, in a non-limiting example, an autoencoder 1010 including a diffusion model 1013 may be a model trained to learn a latent representation of graph structure data 1001 and generate new data.

In an example, the autoencoder 1010 may also include a graph encoder 1011 and a decoder 1012. The graph encoder 1011 may be an encoder that generates a node-specific latent vector 1014, which is the encoding data of the graph structure data 1001, from the graph structure data 1001 as described above. For example, the graph encoder 1011 may include the graph encoder 710 described above with reference to FIG. 7.

The decoder 1012 may be trained to output restored features 1002 of action data corresponding to the graph structure data 1001 from the node-specific latent vector 1014 output from the graph encoder 1011. The restored features 1002 of action data may include features extracted from the action data included in a synthesis process corresponding to the graph structure data 1001. As an example, the node-specific latent vector 1014 of the graph structure data may include features of each node of the graph structure data 1001 updated by a graph convolution operation. For example, the decoder 1012 may be trained to output the restored features 1002 of action data included in the synthesis process.

As described above, the autoencoder 1010 may include the diffusion model 1013 and the diffusion model 1013 may be trained to restore the node-specific latent vector 1014 from Gaussian noise.

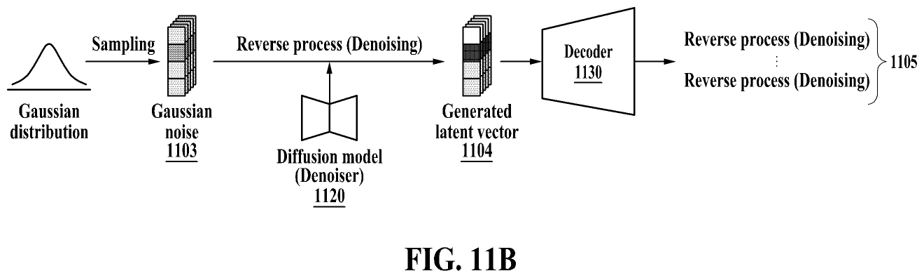

FIGS. 11A and 11B illustrate an examples of an autoencoder with a diffusion model for generating features of new action data according to one or more embodiments.

Referring to FIG. 11A, in a non-limiting example, a node-specific latent vector X₀ 1101 may be converted into Gaussian noise XT 1102 through a forward process. In a reverse process, a diffusion model (or denoiser) 1110 may be trained to restore the node-specific latent vector X₀ 1101 from the Gaussian noise XT 1102.

Referring again to FIG. 10, the trained decoder 1012 may be trained to generate the features 1003 of new action data. A set of features 1003 of new action data generated in the decoder 1012 may correspond to a new synthesis process.

Referring to FIG. 11B, in a non-limiting example, if Gaussian noise 1103, which was randomly extracted from a Gaussian distribution, is input to a trained diffusion model 1120, a node-specific latent vector 1104 may be generated (i.e., a generated latent vector). A trained decoder 1130 may receive the node-specific latent vector 1104 generated from the trained diffusion model 1120 as an input and may generate features 1105 of action data corresponding to the new synthesis process. A set of features 1105 of action data generated by the decoder 1130 may correspond to the new synthesis process.

FIG. 12 illustrates an example electronic device according to one or more embodiments.

Referring to FIG. 12, in a non-limiting example, an electronic device 1200 may include a processor 1201, a memory 1203, and an input/output (I/O) device 1205. The electronic device 1200 may perform a graph structure data processing method corresponding to the synthesis process described above with reference to FIGS. 1 to 11B. For example, the electronic device 1200 may include at least one of a server and a terminal (e.g., a personal computer (PC), a smartphone, a tablet, or a wearable device).

The memory 1203 may include computer-readable instructions. The processor 1201 may be configured to execute computer-readable instructions, such as those stored in the memory 1203, and through execution of the computer-readable instructions, the processor 1201 is configured to perform one or more, or any combination, of the operations and/or methods described herein.

The processor 1201 may be configured to execute programs or applications to configure the processor 1201 to control the electronic apparatus 1200 to perform one or more or all operations and/or methods involving the resolution of a deadlock state and resuming a task, and may include any one or a combination of two or more of, for example, a central processing unit (CPU), a graphic processing unit (GPU), a neural processing unit (NPU) and tensor processing units (TPUs), but is not limited to the above-described examples.

In an example, the processor 1201 may be configured to perform at least one operation of the graph structure data processing method corresponding to the synthesis process described above with reference to FIGS. 1 to 11B.

For example, the processor 1201 may perform at least one of extracting features from action data in chronological order included in the synthesis process, obtaining a vector set corresponding to the action data based on embedding data of features extracted from the action data, generating graph structure data corresponding to the vector set based on time information of the action data, and obtaining encoding data corresponding to the graph structure data based on a graph encoder.

The memory 1203, according to an embodiment may be a volatile memory or a non-volatile memory and may store data regarding the graph structure data processing method corresponding to the synthesis process described above with reference to FIGS. 1 to 11B. For example, the memory 1203 may store data generated during the execution of the graph structure data processing method corresponding to the synthesis process or data required to perform the graph structure data processing method corresponding to the synthesis process. For example, the memory 1203 may store the graph encoder. For example, the memory 1203 may store a property data prediction model (e.g., the property data prediction model 710 of FIG. 7). For example, the memory 1203 may store an autoencoder (e.g., the autoencoder 910 of FIG. 9 or the autoencoder 1010 of FIG. 10).

In an example, the memory 1203 may not be a component of the electronic device 1200 but may be included in an external device accessible by electronic device 1200. In this case, the electronic device 1200 may receive data stored in the memory 1203 included in the external device through a communication device and may transmit data to be stored in the memory 1203.

In an example, the memory 1203 may store a program for implementing the graph structure data processing method corresponding to the synthesis process described above with reference to FIGS. 1 to 11B. The processor 1201 may execute a program stored in the memory 1203 and may control the electronic device 1200. Code of the program executed by the processor 1201 may be stored in the memory 1203.

In an example, the memory 1203 may store instructions, in which the instructions, when executed by the processor 1201, may cause the electronic device 1200 to extract features from the action data in chronological order included in the synthesis process, obtain a vector set corresponding to the action data based on embedding data of the features extracted from the action data, generate graph structure data corresponding to the vector set based on time information of the action data, and obtain encoding data corresponding to the graph structure data based on the graph encoder.

In an example, the electronic device 1200 may include the I/O device 1205 including an input device and an output device. The electronic device 1200 may be connected to an external device (e.g., a PC or a network) through the I/O device 1205 and may exchange data with the external device. For example, the electronic device 1200 may receive synthesis process data through the I/O device 1205 and may output property data, which is an output of graph structure data processing corresponding to the synthesis process, or features of the generated action data.

The electronic device 1200 may further include other components not shown in the drawings. For example, the electronic device 1200 may include a communication device. The communication device may provide a function for the electronic device 1200 to communicate with other electronic devices or other servers over a network. In addition, for example, the electronic device 1200 may further include other components such as a transceiver, various sensors, and a database.

The electronic devices, neural networks, processors, memories, encoders, decoders, graph encoder 250, graph encoder 710, property data prediction model 810, graph encoder 811, decoder 812, autoencoder 910, graph encoder 911, decoder 912, autoencoder 1011, graph encoder 1011, diffusion model 1013, decoder 1012, graph electronic device 1200, memory 1203, and processor 1201 described herein and disclosed herein described with respect to FIGS. 1-12 are implemented by or representative of hardware components. As described above, or in addition to the descriptions above, examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. As described above, or in addition to the descriptions above, example hardware components may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-12 that perform the operations described in this application are performed by computing hardware, for example, by one or more processors or computers, implemented as described above implementing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media, and thus, not a signal per se. As described above, or in addition to the descriptions above, examples of a non-transitory computer-readable storage medium include one or more of any of read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and/or any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. A processor-implemented method, the method comprising:
extracting a feature from action data, the action data being in chronological order and included in a synthesis process;
obtaining a vector set corresponding to the action data, based on embedding data of the feature extracted from the action data;
generating graph structure data corresponding to the vector set, based on time information of the action data; and
obtaining encoding data corresponding to the graph structure data.

2. The method of claim 1, wherein the generating the graph structure data comprises:
determining a node corresponding to respective segments of action data; and
determining an edge between determined nodes, based on the time information of the action data.

3. The method of claim 2, wherein the determined node comprises a first node corresponding to first action data, and
wherein a first node feature of the first node comprises a vector corresponding to the first action data.

4. The method of claim 2, wherein the determined edge comprises a first edge, the first edge connecting a first node corresponding to first action data to a second node corresponding to second action data, and
wherein the method further comprises:
determining a first edge feature of the first edge by encoding a difference between first action time information of the first action data and second action time information of the second action data.

5. The method of claim 2, wherein the determined edge comprises a first edge, the first edge connecting a first node corresponding to first action data to a second node corresponding to second action data, and
wherein the method further comprises:
determining a first edge feature of the first edge by encoding a difference between a first action data feature of the first action data and a second action data feature of the second action data.

6. The method of claim 1, wherein the obtaining the encoding data corresponding to the graph structure data comprises:
obtaining a nearby node feature of a nearby node comprised in a nearby node radius and an updated feature of an updated node, the updated node being updated by reflecting a connected node feature of an edge connected to the nearby node, based on a graph convolution operation; and
obtaining the encoding data corresponding to the graph structure data, based on the updated node feature.

7. The method of claim 1, wherein the obtaining the encoding data corresponding to the graph structure data comprises:
obtaining a nearby node feature of a nearby node comprised in a nearby node radius and an updated feature of an updated node, the updated node being updated by reflecting a connected node feature of an edge connected to the nearby node according to weights, based on an attention network; and
obtaining the encoding data corresponding to the graph structure data, based on the updated node feature.

8. The method of claim 1, further comprising:
obtaining property data, the property data being a result from a prediction from the synthesis process, based on the encoding data corresponding to the graph structure data.

9. The method of claim 1, further comprising:
generating an extracted feature extracted from the action data, based on the encoding data corresponding to the graph structure data for nodes corresponding to respective segments of the action data.

10. The method of claim 9, further comprising:
converting the encoding data corresponding to the graph structure data for the nodes into Gaussian noise; and
restoring the Gaussian noise to the encoding data for the nodes.

11. The method of claim 10, wherein the generating is performed by a decoder trained to generate the extracted feature from the action data, based on the encoding data corresponding to the graph structure data for the nodes, and
wherein the restoring is performed by a diffusion model trained to restore the Gaussian noise to the encoding data for the nodes.

12. The method of claim 11, wherein the trained diffusion model is configured to generate a latent vector from the Gaussian noise, and
wherein the trained decoder is configured to generate a feature of the action data corresponding to the synthesis process from the latent vector generated in the trained diffusion model.

13. A non-transitory computer-readable storage medium storing instructions that, when executed by a processor, cause the processor to perform the method of claim 1.

14. An electronic device, comprising:
processors configured to execute instructions; and
a memory storing the instructions, wherein execution of the instructions configures the processors to:
extract a feature from action data, the action data being in chronological order and included in a synthesis process,
obtain a vector set corresponding to the action data, based on embedding data of the feature extracted from the action data,
generate graph structure data corresponding to the vector set, based on time information of the action data, and
obtain encoding data corresponding to the graph structure data.

15. The electronic device of claim 14, wherein the generating the graph structure data comprises:
determining a node corresponding to respective segments of action data; and
determining an edge between determined nodes, based on the time information of the action data.

16. The electronic device of claim 15, wherein the determined node comprises a first node corresponding to first action data, and
wherein a first node feature of the first node comprises a vector corresponding to the first action data.

17. The electronic device of claim 15, wherein the determined edge comprises a first edge, the first edge connecting a first node corresponding to first action data to a second node corresponding to second action data, and
wherein the processors are further configured to:
determine a first edge feature of the first edge by encoding a difference between first action time information of the first action data and second action time information of the second action data.

18. The electronic device of claim 15, wherein the determined edge comprises a first edge, the first edge connecting a first node corresponding to first action data to a second node corresponding to second action data, and
wherein the processors are further configured to:
determine a first edge feature of the first edge by encoding a difference between a first action data feature of the first action data and a second action data feature of the second action data.

19. The electronic device of claim 14, wherein the obtaining the encoding data corresponding to the graph structure data comprises:
obtaining a nearby node feature of a nearby node comprised in a nearby node radius and an updated feature of an updated, the updated node being updated by reflecting a connected feature of an edge connected to the nearby node, based on a graph convolution operation; and
obtaining the encoding data corresponding to the graph structure data, based on the updated node feature.

20. The electronic device of claim 14, wherein the processors are further configured to:
obtain property data, the property data being a result from a prediction from the synthesis process, based on the encoding data corresponding to the graph structure data.
